(19) United States
(12) Patent Application Publication
(10) Pub. No.: US 2004/0265313 A1
(43) Pub. Date: Dec. 30, 2004

(54) Methods to regulate biofilm formation

(75) Inventors: Douglas G. Storey, Calgary (CA); Michael D. Parkins, Calgary (CA); Howard Ceri, Calgary (CA); Merle E. Olson, Calgary (CA)

Correspondence Address: BURNS DOANE SWECKER & MATHIS L L P, POST OFFICE BOX 1404, ALEXANDRIA, VA, 22313-1404, US

(73) Assignee: University Technologies International, Inc., Calgary (CA)

(21) Appl. No.: 10/828,557

(22) Filed: Apr. 21, 2004

Related U.S. Application Data
(60) Provisional application No. 60/465,153, filed on Apr. 23, 2003.

(51) Int. Cl. A61K 39/395

(52) U.S. Cl. 424/146.1

(57) ABSTRACT

This invention relates to methods and compositions to regulate biofilm formation. In particular, the invention relates to regulation of biofilm formation by modulating the GacA/GacS regulatory system.

### RELATED APPLICATION

This application claims priority under 35 U.S.C. § 119 to U.S. Provisional Application No. 60/465,153 entitled Methods to Regulate Biofilm Formation and filed on Apr. 23, 2003 which is incorporated by reference in its entirety.

### FIELD OF THE INVENTION

This invention relates to methods and compositions to regulate biofilm formation. In particular, the invention relates to regulation of biofilm formation by modulating the GacA/GacS regulatory system.

### REFERENCES

The publications, patents and patent applications referenced herein or in the attachments are incorporated by reference in their entirety to the same extent as if the disclosure of each individual publication, patent application or patent was specifically and individually indicated to be incorporated by reference in its entirety.

### BACKGROUND OF THE INVENTION

Biofilms are an alternate mode of bacterial growth where cells exist within a complex and highly heterogeneous matrix of extracellular polymers adherent to a surface. Pathogenic microbial biofilms display decreased susceptibility to antimicrobial agents and elevated resistance to host immune response, often causing chronic infections. Pseudomonas aeruginosa, a gram negative opportunistic pathogen, forms biofilms within the lungs of cystic fibrosis patients and has become the model organism for the study of biofilm physiology. P. aeruginosa utilizes several global regulatory elements to control expression of its vast array of virulence factors. In P. aeruginosa, the GacA/GacS regulon has been shown to include genes which affect production of pyocyanin, cyanide, lipase, PAI-2 and is essential for virulence in three independent models of infection.

However, studies in other organisms such as fluorescent pseudomonades, have implicated much broader ranging effects of the GacA/GacS regulon. In Pseudomonas chlororaphis O6, which is an aggressive colonizer of plant roots under competitive soil conditions, the GacA/GacS two component regulatory system has been demonstrated to control expression of protease, phytotoxins, and secondary metabolites. P. chlororaphis O6 inhibits growth of several fungal pathogens in vitro. The O6 mutant L21, generated by transposon mutagenesis, lacked production of antifungal phenazines. The O6 gacS gene, encoding a sensor kinase, complemented L21, although the Tn5 insertion site was in gene, ppx encoding exopolyphosphatase. O6 gacS mutants, like L21, lacked in vitro production of phenazines, protease, and HSLs. Confocal laser microscopy, revealed that wild-type O6 but not the gacS mutant produced phenazines on bean roots. The gacS mutant had decreased catalase activity and was less competitive than wild-type in colonization of bean roots in the presence of competing microbes. These findings directly demonstrated a role of gacS in root colonization.

### SUMMARY OF THE INVENTION

The invention relates to the unexpected discovery of the role of the GacA/GacS two component global regulatory system in biofilm formation of both the opportunistic pathogen Pseudomonas aeruginosa and the fluorescent pseudomonad Pseudomonas chlororaphis O6. We have found that the GacA/GacS two component regulatory system is a genetic element necessary for biofilm formation in these pseudomonades. Biofilm growth curves demonstrated that when the response regulator, gacA, was disrupted in P. aeruginosa strain PA14 a 10 fold reduction in biofilm formation capacity resulted relative to wild type PA14 and a toxA derivative. However, no significant difference in the planktonic growth rate of PA14 gacA was observed. Scanning electron microscopy of biofilms formed by PA14 gacA revealed diffuse clusters of cells which failed to aggregate into microcolonies, implying a deficit in biofilm maturation. Twitching motility assays, and PAI-1 autoinducer bioassays reveal normal zones of twitching motility and PAI-1 production, indicating this is not merely an upstream effect on either the las quorum sensing system or type IV pili biogenesis. Antibiotic susceptibility profiling has demonstrated PA14 gacA biofilms have moderately decreased resistance to azythromycin, chloramphenicol, erythromycin, piperacillin, and polymixin B relative to either PA14 wild type or the toxA control. This study establishes the GacA/GacS two component regulatory system as an independent regulatory element in P. aeruginosa biofilm formation. We also demonstrate that the regulatory gacS gene plays an important role in biofilm formation and structure in Pseudomonas chlororaphis O6 (PcO6) using a gacS knock-out mutant generated in PcO6 by Tn-5 insertion. The ability of wild type and mutant strains to form biofilms was evaluated in vitro using the MBEC device. Biofilm formation by the gacS mutant, as evaluated by colony counts and scanning electron microscopy was greatly reduced in comparison with the wild type strain, but it was restored by complementation with an active gacS construct. Given the fact of the gacS involvement in root colonization, our results suggest a plausible role of biofilm formation in PcO6 biocontrol capability.

### BRIEF DESCRIPTION OF THE DRAWINGS

FIGS. 1-3 and 5 show the growth curves of various P. aeruginosa strains.

FIG. 4 provides scanning electron micrographs of various P. aeruginosa strains.

FIG. 6 illustrates motility assay results of various P. aeruginosa strains.

FIG. 7 illustrates the production of PAI-1 by various P. aeruginosa strains.

FIG. 8 illustrates P. chlororaphis O6 biofilm growth on MBEC device:

(A) a wild type;

(B) a gacS knock-out mutant; and

(C) a gacS/+-complemented mutant.

FIGS. 9 and 10 provide scanning electron micrographs of P. chlororaphis O6 strains at different cell densities.

A and B represent wild type P. chlororaphis at different magnifications showing dense biofilm formation, organization into a microcolony three-dimensional structure typical of biofilm formation.

C and D represent different magnifications of SEMs of the gacS mutant showing sparse cell attachment and failure to generate microcolony formation, but rather clusters of small cell groupings with little organized structure.

E and F are different magnifications of SEMs of the gacS mutant complemented with the gacS gene in trans. Formation of true biofilm structure returned to the mutant by restoration of an active gacS gene as seen by the microcolony organization into complex architecture typical of a biofilm.

Magnification for pictures:

Wild type, A=1.1 K, B=3.5 K;

Mutant C=1.5 K; D=3.5 K;

Complemented strain: E=1.0 K and F=3.5 K.

### DETAILED DESCRIPTION OF THE INVENTION

In the first embodiment, the present invention is directed to methods of inhibition of biofilm formation by pathogenic bacteria and described in detail in Attachments A, B, C and D, the entire content of each of which is hereby incorporated by reference.

In addition to Pseudomonas aeruginosa, many other organisms were also found to contain proteins bearing high levels of sequence identity to GacA. It is contemplated that inhibitors, antagonists or antibodies of the GacA/GacS regulatory system can also be used to inhibit biofilm formation of, and to treat diseases associated with, other organisms as well. Proteins which are homologous to GacA and the organisms which contain these proteins can be found by sequence homology searches known in the art. In particular, the following are examples of proteins which have a sequence identity of at least 25% with GacA:

| | | Sequence |
|---|---|---|
| Organism | Protein | Identity |
| Pseudomonas | GacA | 100% |
| aeruginosa | | |
| P. viridiflava | RepB | 89% |
| P. syringae | cognate response regulator gacA | 89% |
| P. syringae | fix J-like response regulator | 89% |
| P. fluorescense | response regulator (AF065156) | 87% |
| P. fluorescense | response regulator/transcription | 86% |
| | activator (L29642) | |
| P. fluorescense | gacA (M80913) | 86% |
| V. cholerae | transcription regulator luxR family | 62% |
| E. coli 0157:H7 | | 60% |
| E. coli | UVRY protein | 60% |
| Salmonella | SirA | 60% |
| Typhimurium | | |
| Erwinia carotovora | expA | 59% |
| Xylella fasticliosci | luxR/uhpA | 43% |
| Streptomyces coe | two-component response regulator | 40% |
| Deinococcus | | 38% |
| radiodurans | | |
| P. Solonacearum | vsrD protein | 37% |
| Ralstonia | vsrD protein | 37% |
| solanacearum | | |
| V. cholerae | transcription regulator LuxR family | 37% |
| | VC1277 | |
| P. aeruginosa | nitrate/nitrite regulatory protein | 36% |
| P. aeruginosa | two-component response regulator | 36% |
| | NarL | |
| Streptomyces | A3(2) (AL355774) | 36% |
| coelicolor | | |
| Neisse meningitidis | transcriptional regulator, LuxR family | 34% |
| Deinococus | DNA-binding response regulator | 34% |
| radiodurans | | |
| P. aeruginosa | two-component response regulator | 34% |
| | PA3045 | |
| Streptomyces | putative response regulator | 34% |
| coelicolor | | |
| Streptococcus | response regulator | 32% |
| pneumoniae | | |
| S. coelicolor | A3(2) (AL049754) | 33% |
| B. subtilis | [yvqe] homolog yvqc | 32% |
| Bacillus h. | two-component regulator | 33% |
| P. aeruginosa | two-component regulator PA0601 | 34% |
| Streptomyces | A3 | 34% |
| coelicolor | | |
| Lactococcus lactis | RrD | 34% |
| Synechocystis sp. | nitrate/nitrite response regulator | 34% |
| | protein | |
| Streptomyces | response regulator | 32% |
| coelicolor | | |
| Bordetella pertussis | bvgA | 34% |
| Bordetella | bvgA | 34% |
| bronchiseptica | | |
| Bordetella | bvgA | 34% |
| parapertussis | | |
| Erwinia amy | HrpY | 30% |
| Staphylococcus aureus | response regulator | 31% |
| Deinococcus | DNA-binding response regulator | 33% |
| radiodurans | | |
| P. Stutzeri | NarL protein | 32% |
| Bacillus h. | response regulator | 31% |
| Bacallus subtilis | yfik | 29% |
| Bacillus brevis | DEGU regulatory protein | 27% |
| Bacillus halodurans | two-component response regulator | 27% |
| Bacillus subtilis | DEGU, extracellular proteinase | 26% |
| | response regulator | |

In the second embodiment, the present invention provides methods of regulation of biofilm formation by symbiotic bacteria, for example, plant root bacteria. It is contemplated that activators, inhibitors, agonists, antagonists or antibodies of the GacA/GacS regulatory system can also be used to regulate biofilm formation of, and to provide regulation of symbiotic bacteria -host interaction.

For example, Pseudomonas chlororaphis O6 (PcO6) is an aggressive colonizer of plant roots under competitive soil conditions. Root colonization by PcO6 induces foliar resistance to Pseudomonas syringae pv. tabaci in tobacco. To understand the genes involved in root colonization, mutations were generated in O6 by Tn-5 insertion. One mutant was complemented in phenotype by the gacS gene. The gacS knock-out mutant was deficient in phenazine, acyl homoserine lactones and extracellular protease production. The ability of wild type and mutant strains to form biofilms was evaluated in vitro using the MBEC device. Biofilm formation by the gacS mutant, as evaluated by colony counts and SEM was greatly reduced, but it was restored by complementation with an active gacS construct. The results demonstrate that the regulatory gacS gene plays an important role in biofilm formation and structure in PcO6, which may play a role in its biocontrol capability.

### EXAMPLES

### Example 1

Growth conditions of Pseudomonas Chlororaphis O6

Pseudomonas chlororaphis O6 wild type strain was isolated from roots of wheat plants grown in Logan, Utah, USA (4). P. chlororaphis O6 knockout gacS mutant strain and gacS complemented strain were generated in (3). Bacteria were grown in 5.0 mL of King's medium (KB) (Protease peptone #3(Difco)-20 g, KH₂PO₄-1.5 g, MgSO₄7H₂O-1.5 g, Glycerol-15.0 mL per L) at room temperature (18-22° C.) with shaking at 120 rpm, on in King's B agar plates at 28° C. Growth of the anticipated bacteria was noted: orange colonies on KB plates for wild type strain, colorless colonies of the gacS mutant on KB plus kanamycin (25 μg/ml) and orange colonies on KB plus kanamycin and tetracycline (25 μg/ml) of the complemented mutant. Biofilms were grown in the MBEC device following standard methodology described in (1) and (2).

### Example 2

Scanning Electron Microscopy

After 24 h, pegs were removed from the 96-peg lid of the MBEC device and air dried for 1-2 h at room temperature, under a fume hood. Samples were fixed in 5% glutaraldehyde prepared in 0.1 M sodium cacodylate buffer, pH 7.2, at room temperature. After fixation, pegs were allowed to dry overnight on a Petri-dish, then assembled onto stubs and sputter-coated with gold-palladium. Scanning electron microscopy was performed using a Cambridge Model 360 SEM at 20 kv emission. Digital images were captured from the SEM using OmniVision (v. 5.1) software.

### Example 3

Growth Conditions, Sample Analysis and Bio-Assays of Pseudomonas aeruginosa

Biofilm and planktonic growth studies were performed using the Calgary Biofilm Device (CBD) (MBEC™ Biofilm Technologies Limited). Pseudomonas aeruginosa PA14 wild type, gacA and toxA strains were grown for 24 hours in Tryptic Soy Broth (BDH). Biofilm and planktonic populations were sampled at points.

Sampling of biofilm populations was achieved by dislodging a peg from the 96 peg lid, whereas planktonic populations were sampled by removing an aliquot from the growth vessel. Biofilms were disrupted to release individual component cells by sonication. Cell counts of both populations were determined by serial dilution in 0.9% saline and spot plating on Tryptic Soy Agar plates (BDH). Antibiotic susceptibility profiling of P. aeruginosa PA14 wild type, toxA and gacA strains was performed using the MBEC™ device as per manufacturers instructions (MBEC™ Biofilm Technologies Limited).

To assess for alterations in the levels of autoinducer production, bio-assays were performed on P. aeruginosa PA14 wild type, PA14 toxA, and PA14 gacA as described by Pearson et al. (1995) using the reporter strain E. coli MG4 (pKDT17).

To assess for alerations in type IV pili mediated twitching motility of P. aeruginosa PA14 gacA compared to wild type PA14 or the control knock-out strain PA14 toxA, zones of twitching were measured and compared. On very thin LB or TSA plates (<2 mm thick), each of the three PA14 derivative strains were inoculated using a stab loop. Bacterial proliferation between the agar and the plate was measured as the zone of twitching.

Results

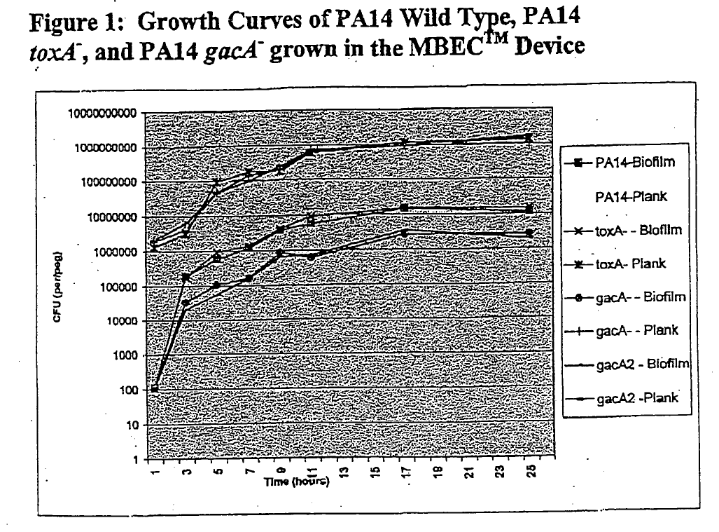

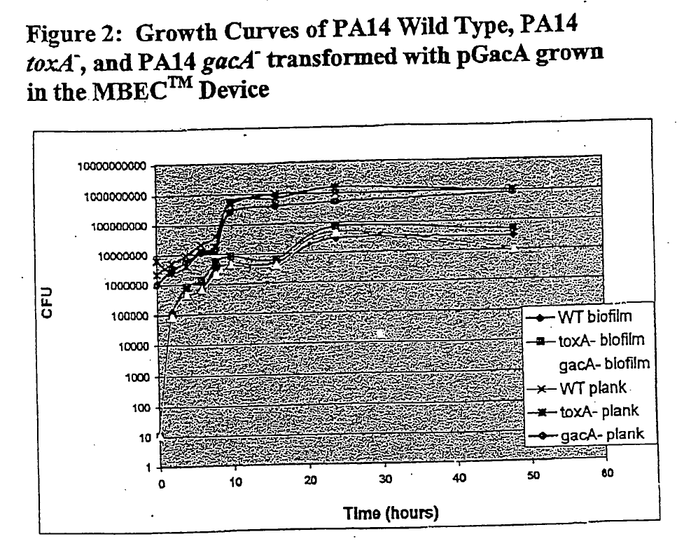

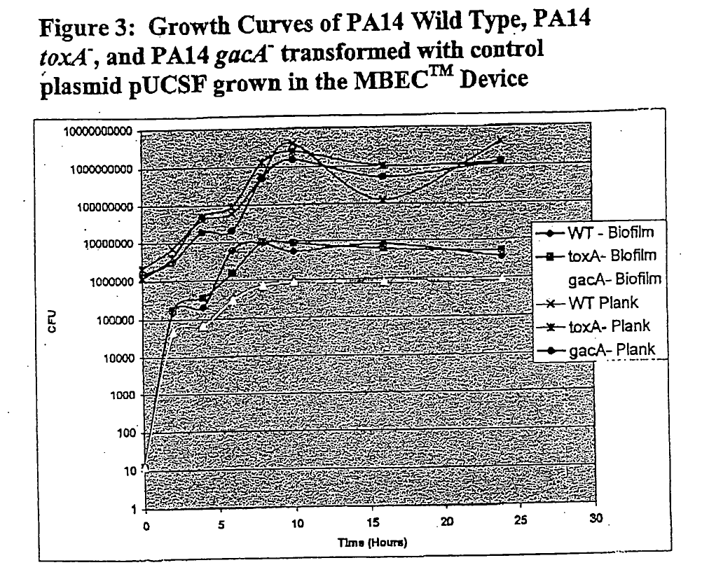

Biofilm growth curves demonstrated that when the response regulator of the two component regulatory system, gacA, was disrupted in P. aeruginosa strain PA14, a 10-fold reduction in biofilm formation ensued relative to wild type PA14 and a toxA derivative. This reduction in biofilm formation was evident in both the rate at which biofilms were formed over a 24 hour time period as well as final biofilm size. However, no significant difference in the planktonic growth rate of PA14 gacA was observed compared to the two control strains (See FIG. 1). When gacA was provided in trans in the multi-copy vector pGacA to strain PA14 gacA, the defect in biofilm formation ability was abrogated (See FIG. 2). The biofilm formation defect was not corrected in PA14 gacA when transformed with the control vector pUCSF (See FIG. 3).

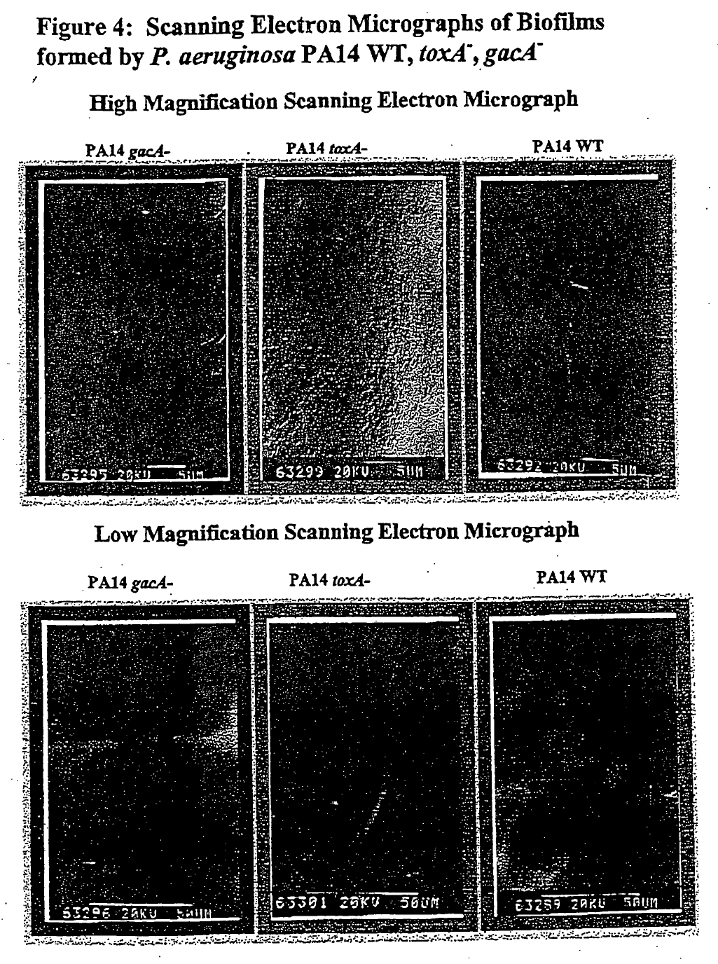

Scanning electron microscopy of biofilms fromed by PA14 gacA revealed diffuse clusters of adherent cells which failed to aggregate into microcolonies. Biofilms formed by wild type PA14 or the control toxA deriviative had normal biofilm characteristics and formed a dense mat of bacterial growth. This evidence implies that the gacA knock-out strain of PA14 has an inherent defect in biofilm maturation, the result of disrupting the GacA/GacS regulon (See FIG. 4).

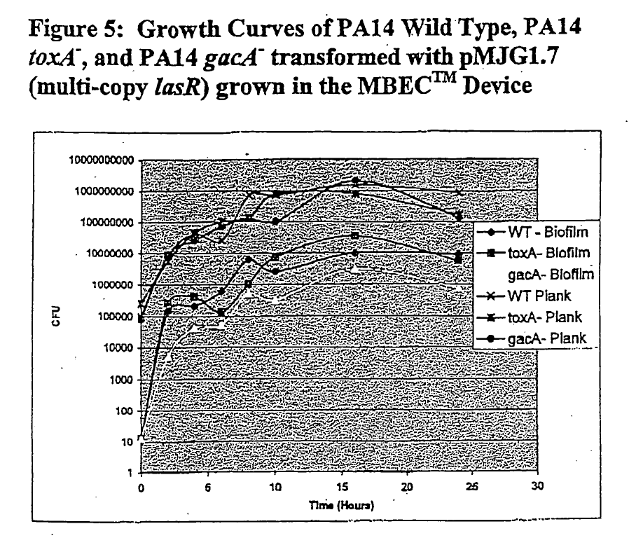

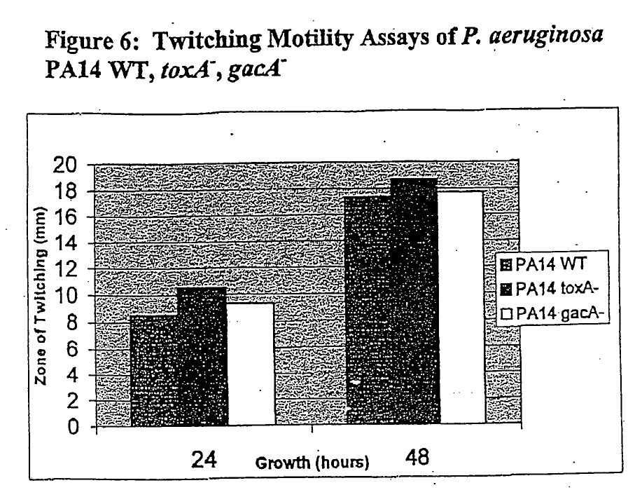

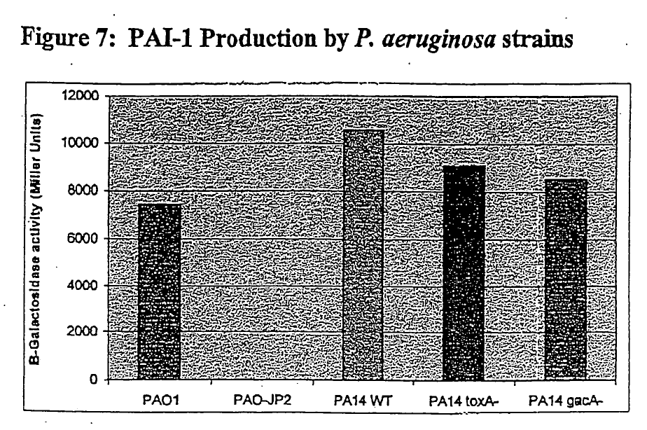

To ensure that the defect in biofilm formation ability caused by the disruption of the GacA/GacS regulon of P. aeruginosa is not merely an upstream effect acting on factors already identified to be involved in biofilm formation, several bioassays were perfomed. Growth curves were perfomed on strains PA14, PA14 toxA and gacA transformed with pMJG1.7, a multi-copy vector expressing lasR. Over-expression of lasR did not complement the biofilm formation defect of strain PA14 gacA (See FIG. 5). LasR is the transcriptional activator of the las quorum sensing system demonstrated to be necessary for biofilm maturation. Twitching motility assays revealed that P. aeruginosa PA14 gacA does not have altered twitching motility mediated by type IV pili relative to either control strains (See FIG. 6). Twitching motility has been shown to be necessary for cellular aggregation to form microcolonies, during the initial steps of biofilm formation. Bioassays used to detect the level of autoinducer production in P. aeruginosa demostrated that PA14 gacA does not have significantly altered levels of N-3-oxododecanoyl-L-homoserine lactone (PAI-1) relative to the two control strains. PAI-1 has been shown to be required for microcolony maturation into fully developed biofilms (See FIG. 7). The results of these studies confirm that the gacA/gacS regulon itself, and not downstream factors previously identified in biofilm formation, is responsible for the biofim formation defect of P. aeruginosa PA14 gacA.

Antibiotic susceptibility profiling has demonstrated PA14 gacA biofilms have moderately decreased resistance to azythromycin, chloramphenicol, erythromycin, piperacillin, and polymixin B relative to either PA14 wild type or the toxA control strain.

These findings clearly demonstrate a role for the GacA/GacS two component regulatory system of P. aeruginosa in biofilm formation. Current studies are underway to determine if the GacA/GacS regulatory system homologs in other pathogenic bacteria similarly play a role in biofilm formation. Disruption of biofilm formation by targeting the GacA/GacS two component regulatory system is being considered as a potential therapeutic treatment for cystic fibrosis pulmonary infections.

Pseudomonas chlororaphis O6

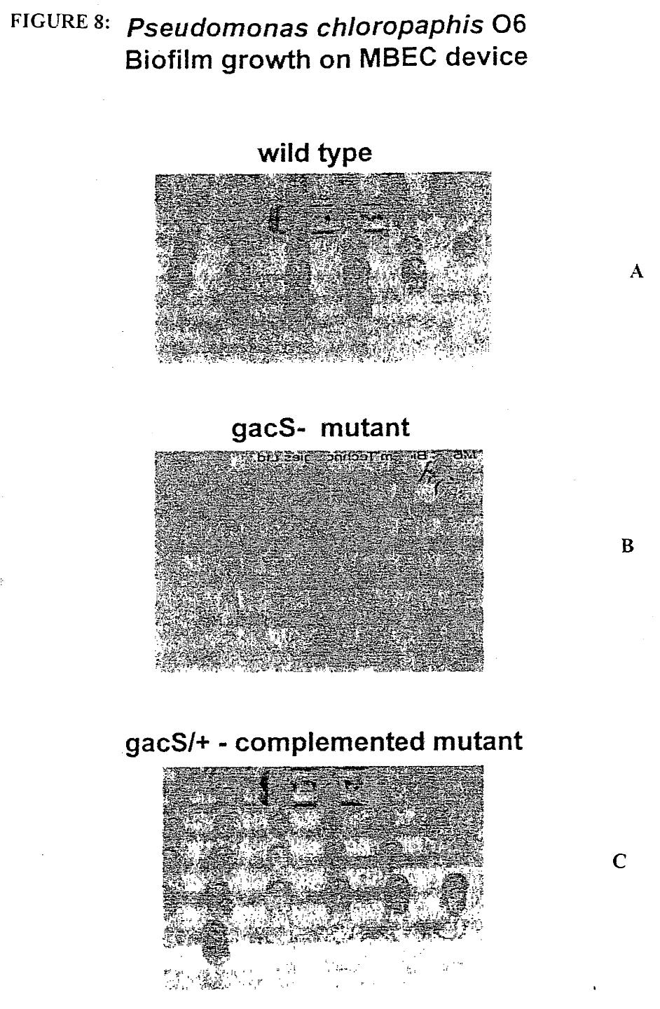

As shown at FIG. 8, when the response regulator of the two component regulatory system, gacS, was disrupted in a gacS knock-out mutant of P. chlororaphis O6, a complete suppression of biofilm formation on MBEC device ensued relative to wild type PcO6. When gacS was provided in trans in the multi-copy vector pGacS to strain PcO6gacS, the defect in biofilm formation ability was abrogated (See FIG. 8).

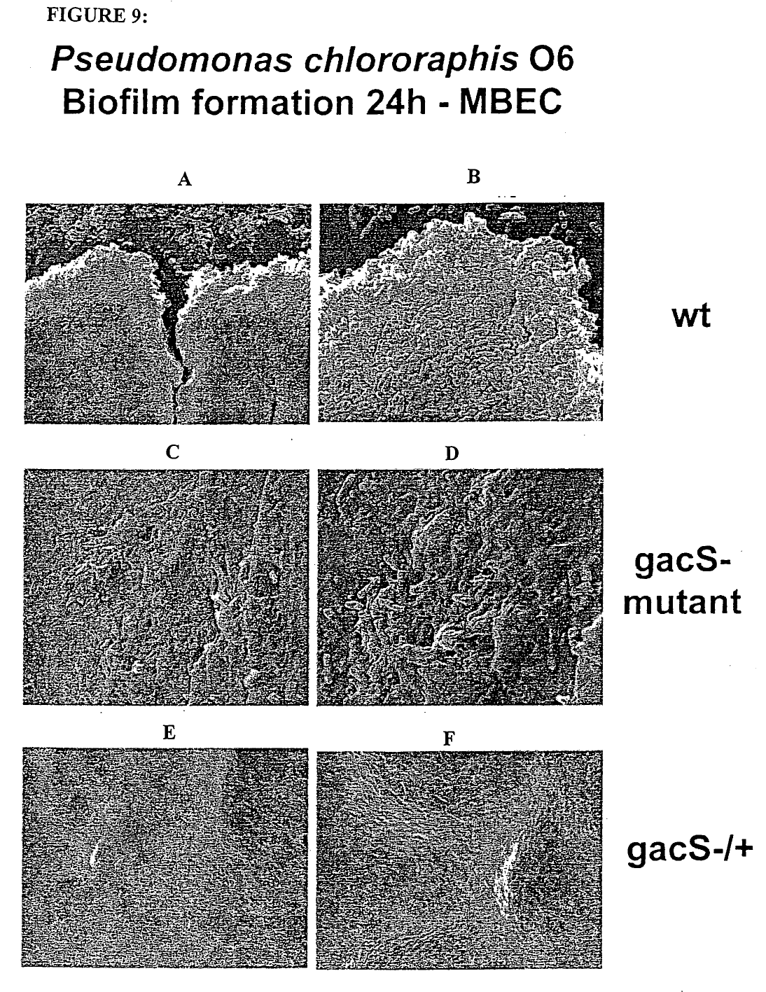

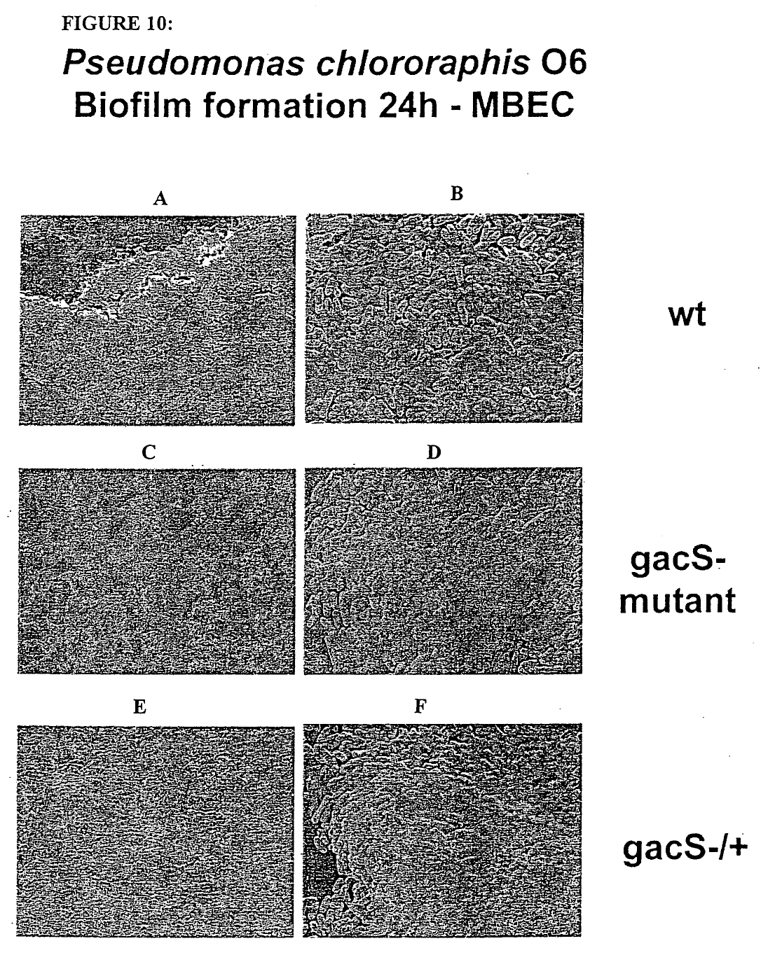

Scanning electron microscopy of biofilms formed by PcO6gacS revealed diffuse clusters of adherent cells which failed to aggregate into microcolonies. (FIGS. 9 and 10 C, D). Biofilms formed by wild type PcO6 (FIGS. 9 and 10 A, B) or gacS/+-complemented mutant (FIGS. 9 and 10 E, F) had normal biofilm characteristics and formed a dense mat of bacterial growth. This evidense implies that a gacS knock-out mutant of P. chlororaphis O6 has an inherent defect in biofilm maturation, the result of disrupting the GacA/GacS regulon.

References

1. Ceri, H.; Olson, M. E.; Stemick, C.; Read, R. R.; Morck, D., and Buret, A. 1999. The Calgary Biofilm Device: A new technology for the rapid determination of antibiotic susceptibility of bacterial biofilms. J. Clin. Microbiol. 37:1771-1776.

2. Ceri, H.; Olson, M.; Morck, D.; Storey, D.; Read, R.; Buret, A.; and Olson, B. 2001. The MBEC Assay System: multiple equivalent biofilms for antibiotic and biocide susceptibility. Methods Enzymol. 337:377-384.

3. Kim, Y. C.; Seong, K. Y.; and Anderson, A. J. 2001 Sensor kinase GacS regulates production of quorum sensing factors, secondary metabolites and root colonization in Pseudomonas chlororaphis O6. Phytopathology 91:S49.

4. Radtke, C.; Cook, W. S. and Anderson, A. J. (1994) Factors affecting antagonism of growth of Phanerochaete chrysosporium by bacteria isolated from soils. Appl. Microbiol. Biotechnol. 41:274-280.

What is claimed is:

1. A method of preventing biofilm formation comprising inhibiting the gacA/gacS regulatory system of an organism.

2. The method of claim 1 wherein the organism is P. aeruginosa.

3. The method of claim 1 wherein the inhibition is produced by antibodies to gacS.

4. A composition useful for preventing biofilm formation comprising a compound which inhibits the gacA/gacS regulatory system in a pharmaceutically acceptable form.

5. The composition of claim 4 wherein the compound is an antibody to gacS.

6. The composition of claim 4 wherein the compound is a small molecule which inhibits gacS

7. A method to treat a biofilm infection in a subject comprising inhibiting the gacA/gacS regulatory system of an organism.

8. The method of claim 7 wherein the organism is P. aeruginosa.

9. The method of claim 7 wherein the inhibition is produced by antibodies to gacS.

10. A method of regulating biofilm formation by an organism comprising modulating the gacA/gacS regulatory system of the organism.

11. The method of claim 10 wherein the organism is a symbiotic bacterium.

12. The method of claim 10 wherein the organism is a plant root bacterium.

13. The method of claim 10 wherein the organism is P. chlororaphis.

14. The method of claim 10 wherein modulating is produced by antibodies to gacS.

15. The method of claim 10 wherein modulating is produced by a small molecule specifically binding with gacS of the organism.

16. A composition useful for regulating biofilm formation by an organism comprising a compound which modulates the gacA/gacS regulatory system of the organism.

17. The composition of claim 15 wherein the compound is an antibody to gacS.

18. The composition of claim 15 wherein the compound is a small molecule specifically binding with gacS of the organism.
